(19) United States
(12) Patent Application Publication
(10) Pub. No.: US 2023/0296580 A1
(43) Pub. Date: Sep. 21, 2023

(54) CYCLIC MISCIBLE HYDROCARBON GAS INJECTION-SOAK-PRODUCTION AND USES THEREOF FOR ENHANCED OIL RECOVERY IN UNCONVENTIONAL RESERVOIRS

(71) Applicants: UNIVERSITY OF WYOMING, Laramie, WY (US); Hess Corporation, Houston, TX (US)

(72) Inventors: Morteza AKBARABADI, Laramie, WY (US); Amir Hossein ALIZADEH, Laramie, WY (US); Mohammad PIRI, Laramie, WY (US); Nagi NAGARAJAN, Houston, TX (US)

(21) Appl. No.: 18/016,634

(22) PCT Filed: Jul. 15, 2021
(86) PCT No.: PCT/US21/41861

§ 371 Date: Jan. 17, 2023

Related U.S. Application Data
(60) Provisional application No. 63/053,053, filed on Jul. 17, 2020.

(51) Int. Cl. G01N 33/24; C09K 8/594

(52) U.S. Cl. CPC G01N 33/241; C09K 8/594

(57) ABSTRACT

The invention is directed to methods for improving the recovery of oil from subterranean geological formations, such as unconventional reservoirs. The invention is also directed to methods for evaluating the efficiency of inject-soak-produce gas injection methods for oil recovery in unconventional reservoirs. In one embodiment, a method for evaluating the efficiency of oil recovery from an unconventional reservoir rock sample includes characterizing the rock sample by imaging the rock sample using a nano-CT scanner and/or scanning electron microscopy (SEM); cleaning the rock sample by flowing solvent through it; saturating the rock sample with crude oil; and establishing initial water saturation in the rock sample.

### CROSS-REFERENCE TO RELATED APPLICATIONS

This application claims the benefit of priority to U.S. Provisional Patent Application No. 63/053053, filed Jul. 17, 2020 which is hereby incorporated by reference in its entirety.

### BACKGROUND OF INVENTION

The present invention is directed to methods for improving the recovery of oil from subterranean geological formations, such as unconventional reservoirs, and methods for evaluating the efficiency of cyclic miscible gas injection for oil recovery in unconventional reservoirs.

Unconventional oil reservoirs, such as shale oil (i.e., a porous rock hosting significant amounts of oil and gas), have become a major source of hydrocarbon production in the United States. To meet global energy demands, there has been a drive to improve oil recovery from these unconventional oil reservoirs. One method that has been used to enhance oil recovery from unconventional oil reservoirs is miscible carbon dioxide (CO₂) injection (see Kovscek et al., Experimental investigation of oil recovery from siliceous shale by CO₂ injection, SPE Annual Technical Conference and Exhibition, Sep. 21-24, 2008, Denver, Colorado). However, the cost and unavailability of CO₂ may make the injection of CO₂ impractical (see Kovscek et al., 2008; Vega et al., Experimental investigation of oil recovery from siliceous shale by miscible CO₂ injection, SPE Annual Technical Conference and Exhibition, Sep. 19-22, 2010, Florence, Italy; Gamadi et al., An experimental study of cyclic gas injection to improve shale oil recovery, SPE Annual Technical Conference and Exhibition, Sep. 30-Oct. 2, 2013, New Orleans, Louisiana; Gamadi et al., An experimental study of cyclic CO₂ injection to improve shale oil recovery, SPE Improved Oil Recovery Symposium, Apr. 12-16, 2014, Tulsa, Oklahoma; Wilson, Modeling of EOR in shale reservoirs stimulated by cyclic gas injection, J. Petrol. Technol. 2015, 67(01); Wan et al., Evaluation of the EOR potential in hydraulically fractured shale oil reservoirs by cyclic gas injection, Petrol. Sci. and Technol. 2015, 33:7,812-818.) Thus, there remains a need in the oil industry for methods for improving oil recovery in unconventional reservoirs that do not use CO₂ or that minimize the use of CO₂ during gas injection. To ensure the efficient use of resources, there is also a need to test the efficiency of these methods before deployment in the field.

### SUMMARY OF THE INVENTION

The present invention provides a method for enhancing oil recovery from subterranean geological formations, particularly from unconventional reservoirs, that minimizes the use of CO2. The present invention also provides a laboratory method to evaluate methods for enhancing oil recovery.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-B. Examples of X-ray images of Sample A obtained using a nano-CT scanner at a resolution of 64 nm. The images were used to estimate the porosity and pore-size distribution of the sample.

FIG. 2: Pore-size distribution of Sample A estimated using nano-CT images.

FIG. 3: SEM micrographs of salt crystals (light gray minerals) both on the surface and in the pore elements of the rock observed from backscatter-electron (BSE) imaging generated using a TLD in immersion mode at 1 kV and 0.1 nA.

FIG. 4: Schematic diagram of the core-flooding setup used in Examples 1-3.

FIGS. 5A-B: Oil production from Samples (FIGS. 5A) A and (FIG. 5B) B during spontaneous imbibition of brine. In FIG. 5B, imbibition time: (a) 0, (b) 1470 min, (c) 3240 min, (d) 5930 min, (e) 7460 min, and (f) 8640 min.

FIG. 6: The volume of oil production from Sample B versus time during spontaneous imbibition of brine.

FIG. 7: The volume of oil produced from Samples BA, BB, CA, and CB versus time during spontaneous imbibition of brine. Samples CA and CB were stored in crude oil after reaching the desired initial water saturation, which resulted in imbibition of some oil into each and slight reduction in their initial water saturations.

FIGS. 8A-F: Preparation of the composite core: (FIG. 8A) one piece of the plug was covered with several layers of proppants, (FIG. 8B) and (FIG. 8C) the second piece was placed on the first and the core sample was wrapped with Teflon tape, (FIG. 8D) the core samples were placed together, (FIG. 8E) the composite core along with the core holder's end pieces were wrapped with shrink tube, and (FIG. 8F) the shrink tube was wrapped with Aluminum tape.

FIG. 9: Variations of inlet/outlet pressures of the core with time from the beginning of the first cycle to the end of the blow-down process in Example 1.

FIG. 10: Variations of pressure drop with time during the production step of the first cycle in Example 2.

FIG. 11: Photo of the separator (visual cell) taken at the end of the third cycle before the blow-down process in Example 2.

FIG. 12: Variations of inlet/outlet pressures of the core with time from the beginning of the first cycle to the end of the blow-down process in Example 2.

FIGS. 13A-C: Photos of the separator (visual cell) taken during the blow-down process in Example 2 at the separator pressures of (FIG. 13A) 20.68, (FIG. 13B) 13.79, and (FIG. 13C) 6.89 MPa.

FIGS. 14A-D: Photos of the separator (visual cell) in Example 3 at the end of (FIG. 14A) first, (FIG. 14B) second, (FIG. 14C) third, and (FIG. 14D) fourth cycles.

FIG. 15: Variations of inlet/outlet pressures of the core with time from the beginning of the first cycle to the end of the blow-down process in Example 3.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

In general, the terms and phrases used herein have their art-recognized meaning, which can be found by reference to standard texts, journal references and contexts known to those skilled in the art. The following definitions are provided to clarify their specific use in the context of the invention.

To meet global energy demand, there has been a drive to enhance oil recovery from unconventional reservoirs. To this end, previous methods have faced challenges. For example, although miscible CO₂ injection has been used to enhance oil recovery, its large-scale deployment can be impractical in some regions because of CO₂ costs and unavailability.

The inventors have developed a method to enhance oil recovery from subterranean geological formations, particularly unconventional reservoirs, that minimizes the use of CO₂. The inventors have also developed methods for laboratory evaluation of the invented method to enhance oil recovery.

The invention may be understood more readily by reference to the following detailed description of exemplary embodiments of the invention and the examples included therein. It is to be understood that this invention is not limited by these exemplary embodiments or examples. It is also to be understood that the terminology used herein is for the purpose of describing particular embodiments only and is not intended to be limiting.

As used herein, the term “unconventional reservoir” refers to a petroleum reservoir with a permeability/viscosity ratio that requires the use of technology to alter either rock permeability or fluid viscosity to produce petroleum at a commercially competitive rate. Examples of unconventional reservoirs are Permian and Eagle Ford.

The term “porous rock” refers to a rock that has pores and can store fluids. Examples of porous rock include sandstone and carbonate.

The term “ambient conditions” refers to conditions wherein the temperature is room temperature and the pressure is atmospheric pressure. Room temperature ranges between 15° C. and 30° C., preferably between 20° C. and 25° C.

Atmospheric pressure ranges between 735 mm Hg and 785 mm Hg. Preferably, atmospheric pressure is 760 mm Hg.

The term “reservoir conditions” refers to conditions wherein the temperature and pressure reflect the temperature and pressure of a target reservoir. The temperature and pressure of the target reservoir varies as a function of the reservoir's proximity to the earth's mantle and the composition of the porous medium containing fluid (e.g., oil). Reservoir temperatures and pressures can be determined by methods known in the art.

The term “pore volume” refers to the volume of void spaces in any porous material. Pore volumes can be determined by methods known in the art (e.g., from the bulk volume of a core sample and the measured porosity of the sample).

The term “net confining pressure” refers to the difference between the pore pressure and the overburden pressure implemented around a core sample.

The terms “stock tank oil” and “dead oil” can be used interchangeably and refer to oil that releases no dissolved gas at ambient condition and that is, for example, obtained from a separator in which the live oil was flashed to ambient conditions.

The term “live oil” refers to an oil that comprises dissolved gases, which may be released from solution at surface conditions.

The term “huff-n-puff” (also referred to herein as “inject-soak-produce”) refers to a cyclic process wherein a liquid or gas is injected into an oil reservoir (e.g., an unconventional reservoir) and then the reservoir is soaked with the injected liquid or gas. After soaking, the oil reservoir is put to production. Production fluids may be produced from the same side as the gas injection side and/or from the opposite side as the gas injection side.

The term “flow-through technique” refers to a cleaning process to extract salt precipitates from a rock sample wherein a solvent is flowed through the sample. In some embodiments, a flow-through cleaning method comprises flowing a predetermined volume of solvent through the sample. In other embodiments, the solvent effluent from the rock sample may be monitored (e.g., continuously monitored or periodically monitored) during the flow-through process for salt concentration. Once the salt concentration has fallen below a predetermined threshold, the sample may be considered sufficiently cleaned. In one embodiment, the predetermined salt threshold is approximately 5,000 ppm. Optionally, the flow-through cleaning process may further include flushing the sample with gas, such as Helium to remove solvent from the sample. Optionally, the flow-through cleaning process may further include placing the sample under vacuum and/or oven drying.

The term “initial water saturation” S_{wi} refers to the portion of the interconnected pore volume of a rock sample which is occupied by water, expressed as a percent. Initial water saturation (S_{wi}) may be determined by methods well known and understood in the art. In an embodiment, for example, initial water saturation (S_{wi}) is determined by first saturating the rock sample with brine and then displacing brine with oil to determine initial water saturation.

Examples of “flow-through techniques” and methods for measuring “initial water saturation” (Swi) are provided in the following references, the contents of which are hereby incorporated by reference:

a) M. Khishvand, A. H. Alizadeh, I. Oraki Kohshour, M. Piri, and R. S. Prasad, In-situ characterization of wettability alteration and displacement mechanisms governing recovery enhancement due to low-salinity waterflooding, Water Resources Research, 53:1-17 DOI: 10.1002/2016WR020191 (2017);

b) G. Kelechi Ekechukwu, M. Khishvand, W. Kuang, M. Piri, and S. Masalmeh, The effect of wettability on waterflood oil recovery in carbonate rock samples: A systematic multi-scale experimental investigation, Transport in Porous Media, 138: 369-400 (2021);

c) H. Alizadeh and M. Piri, The effect of saturation history on three-phase relative permeability: an experimental study, Water Resources Research, 50(2): 1636-1664, DOI:10.1002/2013WR014914 (2014); and

d) M. Akbarabadi and M. Piri, Relative permeability hysteresis and capillary trapping characteristics of supercritical CO2/brine systems: An experimental study at reservoir conditions, Advances in Water Resources, 52: 190-206 (2013).

The invention provides a method for evaluating the efficiency of oil recovery from unconventional reservoirs using gas injection in the laboratory. This method comprises the steps of: (1) scanning a reservoir rock sample; (2) characterizing the reservoir rock sample; (3) cleaning the reservoir rock sample; (4) saturating the reservoir rock sample with crude oil; and (5) establishing initial brine saturation in the reservoir rock sample.

In one embodiment, the reservoir rock sample is scanned using a tomography device that can produce cross-sectional images representing a thickness of less than 4 mm. Suitable examples of a tomography device include an X-ray macro-CT scanner.

In one embodiment, the reservoir rock sample is characterized by imaging it using a nano-CT scanner and/or scanning electron microscopy (SEM). In one embodiment, the reservoir rock sample is further characterized by visualizing and processing the images obtained from the nano-CT scanner and/or SEM using visualization software packages. Examples of suitable visualization software include AVIZO (Thermo Fisher Scientific).

In one embodiment, the reservoir rock sample is cleaned using solvents that can remove the precipitated salts. Examples of suitable solvents include methanol, isopropanol, toluene, and mixtures thereof (e.g., a methanol/toluene mixture). Cleaning the reservoir rock sample before evaluating the efficiency of oil recovery from the reservoir rock sample is important because salt precipitation in reservoir rock pores may alter the hydraulic connectivity of the pore network. Cleaning can be done at moderate pressure and temperature conditions. In one embodiment, cleaning is performed at 35° C. to 95° C. In one embodiment, cleaning is done at 40° C. to 90° C. In one embodiment, cleaning is done at a temperature of 45° C. to 85° C. In one embodiment, cleaning is done at a temperature of 50° C. to 80° C. In one embodiment, cleaning is done at a pressure of 1 MPa to 10 MPa. In one embodiment, cleaning is done at a pressure of 2 MPa to 9 MPa. In one embodiment, cleaning is done at a pressure of 3 MPa to 8 MPa.

In one embodiment, the reservoir rock sample is saturated with crude oil. To saturate the reservoir rock sample with crude oil, the reservoir rock sample is subjected to vacuum and then crude oil is injected into the reservoir rock sample with a low flow rate. In one embodiment, the low flow rate is 0.0001 cm³/min to 0.1 cm³/min. In one embodiment, the low flow rate is 0.0005 cm³/min to 0.05 cm³/min. In one embodiment, the low flow rate is 0.001 cm³/min to 0.0025 cm³/min. In one embodiment, the low flow rate is 0.001 cm³/min. The pressure drop across the core and the amount of oil recovered from the effluent are monitored. In one embodiment, when stable pressure is achieved, and the volume of recovered oil is similar to the volume of injected oil, the oil flow rate is increased until no extra pore elements may be occupied by crude oil. In one embodiment, the reservoir rock sample is saturated with crude oil at low temperature and moderate back pressure. In one embodiment, the reservoir rock sample is saturated with crude oil at a temperature of 18° C. to 60° C. In one embodiment, the reservoir rock sample is saturated with crude oil at a temperature of 20° C. to 55° C. In one embodiment, the reservoir rock sample is saturated with crude oil at a temperature of 25° C. to 50° C. In one embodiment, the reservoir rock sample is saturated with crude oil at a pressure of 1 MPa to 10 MPa. In one embodiment, the reservoir rock sample is saturated with crude oil at a pressure of 2 MPa to 9 MPa. In one embodiment, the reservoir rock sample is saturated with crude oil at a pressure of 3 MPa to 8 MPa.

In one embodiment, the initial water saturation is established by immersing reservoir rock samples in brine (e.g., Samples A and B, as discussed in the examples below) and performing spontaneous imbibition.

In one embodiment, the method for evaluating the efficiency of oil recovery from unconventional reservoirs using gas injection further comprises the step of (6) performing huff-n-puff gas injection (also referred to herein as an inject-soak-produce procedure) on the reservoir rock sample.

In one embodiment, the huff-n-puff gas injection step on the reservoir rock sample is performed by (a) injecting gas into the reservoir rock sample; (b) providing a time period to soak the reservoir rock sample with the injected gas and the fluids (e.g., oil and brine) present in the rock sample; (c) producing production fluids, such as oil, water, and gas; and (d) optionally repeating steps (a) through (c). The injected gas and the fluids (e.g., oil and brine) present in the rock sample are collectively referred to as the reservoir rock sample's fluid contents. Suitable examples of gas that are injected into the reservoir rock sample include methane, ethane, propane, n-butane, isobutane, and mixtures thereof. The gas that is injected into the reservoir rock sample may optionally further include CO₂. The gas that is injected into the reservoir rock sample may include trace amounts of other components, such as nitrogen, pentane, hexane, heptane, octane, nonane, hydrocarbons with ten or more carbon atoms, and mixtures thereof. In one embodiment, the time period for soaking the reservoir rock sample with its fluid contents is 1 h to 300 h. In one embodiment, the time period for soaking the reservoir rock sample with its fluid contents is 5 h to 250 h. In one embodiment, the time period for soaking the reservoir rock sample with its fluid contents is 10 h to 200 h. In one embodiment, the time period for soaking the reservoir rock sample with its fluid contents is 15 h to 150 h. In one embodiment, the time period for soaking the reservoir rock sample with its fluid contents is 20 h to 100 h. In one embodiment, the time period for soaking reservoir rock sample with its fluid contents is 24 h to 72 h. In one embodiment, production fluids are produced from the same side as the gas injection side. In one embodiment, production fluids are produced from the opposite side as the gas injection side.

In one embodiment, the method for evaluating the efficiency of oil recovery from unconventional reservoirs using gas injection further comprises the steps of (6) fracturing the reservoir rock sample; (7) creating a composite core from reservoir rock samples; and (8) performing huff-n-puff gas injection on the composite core.

In one embodiment, the reservoir rock sample is fractured by cutting the sample (e.g., cutting the sample into two pieces by using a band saw). In one embodiment, the reservoir rock sample is fractured using a Brazilian test, wherein tensile stress is exerted on the reservoir rock sample to break the reservoir rock sample along its length.

In one embodiment, the composite core is created by placing two or more reservoir rock samples together (e.g., Samples B and C). In one embodiment, a fracture in a fractured reservoir rock sample that is used in a composite core is filled with proppant. In one embodiment, each reservoir rock sample is wrapped (e.g., with Teflon tape). In one embodiment, each reservoir rock sample is wrapped (e.g., with Teflon tape) and metal mesh is further placed on one end. Without wishing to be bound by theory, it is contemplated that wrapping each reservoir rock sample and placing metal mesh on one end of each reservoir rock sample helps avoid losing proppants. In one embodiment, a filter paper is placed between two adjacent reservoir rock samples (e.g., Samples B and C). Without wishing to be bound by theory, it is contemplated that placing a filter paper between two reservoir rock samples provides capillary continuity between the pore spaces and reduces large gaps between rocks, which might cause fluid hold-up during flooding of the composite core. In one embodiment, the sides of reservoir rock samples that are not covered with metal mesh are placed together and filter paper is inserted in between the reservoir rock samples. Other methods for compositing rock samples are known in the art, depending on rock characteristics and flow direction.

In one embodiment, the huff-n-puff gas injection process on the composite core is performed by (a) injecting gas into the composite core; (b) providing a time period to soak the composite core with the injected gas and the fluids (e.g., oil and brine) present in the composite core; (c) producing production fluids, such as oil, water, and gas; and (d) optionally repeating steps (a) through (c). The injected gas and the fluids (e.g., oil and brine) present in the composite core are collectively referred to as the composite core's fluid contents. Suitable examples of gas that are injected into the composite core include methane, ethane, propane, n-butane, isobutane, and mixtures thereof. The gas that is injected into the composite core may further optionally include CO₂. The gas that is injected into the composite core may include trace amounts of other components, such as nitrogen, pentane, hexane, heptane, octane, nonane, hydrocarbons with ten or more carbon atoms, and mixtures thereof. In one embodiment, the time period for soaking the composite core in its fluid contents is 1 h to 300 h. In one embodiment, the time period for soaking the gas in the composite core is 5 h to 250 h. In one embodiment, the time period for soaking the composite core in its fluid contents is 10 h to 200 h. In one embodiment, the time period for soaking the composite core in its fluid contents is 15 h to 150 h. In one embodiment, the time period for soaking the composite core in its fluid contents is 20 h to 100 h. In one embodiment, the time period for soaking the composite core in its fluid contents is 24 h to 72 h. In one embodiment, production fluids are produced from the same side as the gas injection side. In one embodiment, production fluids are produced from the opposite side as the gas injection side.

The invention also provides a method for improving the recovery of oil from an unconventional reservoir rock sample, comprising (1) establishing initial oil-brine saturation conditions in the rock sample; (2) injecting a gas phase into the rock sample; (3) providing a time period to soak the rock sample in the injected gas and the fluids (e.g., oil and brine) present in the rock sample; (4) recovering oil using the huff-n-puff approach; (5) stabilizing pressure in pores in the rock sample (“pore pressure”); and (6) optionally repeating steps (2) through (5). The injected gas and the fluids (e.g., oil and brine) present in the unconventional reservoir rock sample are collectively referred to as the rock sample's fluid contents.

In one embodiment, the initial oil-brine saturation conditions in the rock sample are established by first saturating the rock sample with brine and then displacing brine with oil to establish initial water saturation (Swi) at a low temperature and pressure. In another embodiment, the initial oil-brine saturation conditions in the rock sample are established by first saturating a sample from the unconventional reservoir with dead oil and then immersing the rock sample into brine in an imbibition cell to establish Swi at a low temperature and pressure.

In one embodiment, the Swi is established at a temperature of 20° C. to 65° C. In one embodiment, the Swi is established at a temperature of 20° C. to 60° C. In one embodiment, the Swi is established at a temperature of 20° C. to 55° C. In one embodiment, the Swi is established at a temperature of 25° C. to 50° C. In one embodiment, the Swi is established at a pressure of 0.1 MPa (atmospheric pressure) to 10 MPa. In one embodiment, the Swi is established at a pressure of 1 MPa to 9 MPa. In one embodiment, the Swi is established at a pressure of 2 MPa to 8 MPa.

In one embodiment, the gas phase injected into the rock sample is selected from the group comprising methane, ethane, propane, n-butane, isobutane, CO₂ and mixtures thereof.

In one embodiment, the gas phase injected into the rock sample is selected from the group consisting of methane, ethane, propane, n-butane, isobutane, and mixtures thereof.

In one embodiment, the gas phase injected into the rock sample further comprises traces of other components, such as nitrogen, pentane, hexane, heptane, octane, nonane, hydrocarbons with ten or more carbon atoms, and mixtures thereof.

In one embodiment, the gas phase injected into the rock sample further comprises CO₂.

In one embodiment, the gas phase is injected continuously into the rock sample under constant pressure during the soaking period while monitoring the gas flow rate. In another embodiment, gas injection into the rock sample is stopped during the soaking period while monitoring the pressure decline.

In one embodiment, the rock sample is soaked in its fluid contents for 1 h to 300 h. In one embodiment, the rock sample is soaked in its fluid contents for 5 h to 250 h. In one embodiment, the rock sample is soaked in its fluid contents for 10 h to 200 h. In one embodiment, the rock sample is soaked in its fluid contents for 15 h to 150 h. In one embodiment, the rock sample is soaked in its fluid contents for 20 h to 100 h. In one embodiment, the rock sample is soaked in its fluid contents for 24 h to 72 h.

In one embodiment, oil is recovered from the unconventional reservoir rock sample using a huff-n-puff approach. In one embodiment, the production step of the huff-n-puff approach is carried out by decreasing back pressure (outlet pressure) using a back pressure regulation pump to a pre-specified pressure. Pre-specified pressures are determined based on the characteristics of the unconventional reservoir rock sample. A pre-specified pressure is greater than bubble point pressure of the live oil, and the live oil bubble point pressure is the pressure at which the first bubble of gas is released from oil.

In one embodiment, at the end of the production step, when the gas flow is stopped, the pore pressure of the rock sample is monitored until it is stabilized. In one embodiment, at the end of the production step, pore pressure is stabilized while extracting fluids (oil, brine, and gas) from the production line using the back pressure regulation pump under a constant pressure mode. Pore pressure is stabilized when the pressure drop is about zero and the flow rate of the back pressure regulation pump is about zero.

In one embodiment, the method for improving the recovery of oil from an unconventional reservoir rock sample further comprises the step of decreasing pore pressure until oil production is negligible. Negligible oil production occurs when pore pressure is low enough such that an extra drop in pressure does not result in significant oil production or does not result in oil production that is economical.

The invention also provides a method for improving the recovery of oil from a reservoir, comprising (1) injecting a gas phase into the unconventional reservoir; (2) providing a time period to soak the unconventional reservoir with the injected gas and the fluids (e.g., oil and brine) present in the unconventional reservoir; (3) recovering oil using the huff-n-puff approach; (4) stabilizing pressure in the unconventional reservoir (“reservoir pressure”); and (5) optionally repeating steps (1) through (4). The injected gas and the fluids (e.g., oil and brine) present in the unconventional reservoir are collectively referred to as the unconventional reservoir's fluid contents. In some embodiments, the reservoir may be an unconventional reservoir.

In one embodiment, the gas phase injected into the unconventional reservoir is selected from the group consisting of methane, ethane, propane, n-butane, isobutane, mixtures thereof.

In one embodiment, the gas phase injected into the unconventional reservoir further comprises traces of other components, such as nitrogen, pentane, hexane, heptane, octane, nonane, hydrocarbons with ten or more carbon atoms, and mixtures thereof.

In one embodiment, the gas phase injected into the unconventional reservoir further comprises CO₂.

In one embodiment, the gas phase is injected continuously into the unconventional reservoir under constant pressure during the soaking period while monitoring the gas flow rate. In another embodiment, gas injection into the unconventional reservoir is stopped during the soaking period while monitoring the pressure decline.

In one embodiment, the unconventional reservoir is soaked with its fluid contents for 5 days to 35 days. In one embodiment, the unconventional reservoir is soaked with its fluid contents for 10 days to 30 days. In one embodiment, the unconventional reservoir is soaked with its fluid contents for 15 days to 25 days.

In one embodiment, oil is recovered from the unconventional reservoir using a huff-n-puff approach. In one embodiment, the production step of the huff-n-puff approach is carried out by decreasing reservoir pressure to a pre-specified pressure through producing fluids (oil, brine, and gas). Pre-specified pressures are determined based on the characteristics of the unconventional reservoir. A pre-specified pressure is greater than bubble point pressure of the live oil, and the live oil bubble point pressure is the pressure at which the first bubble of gas is released from oil.

In one embodiment, the method for improving the recovery of oil from an unconventional reservoir further comprises the step of decreasing reservoir pressure until oil production is negligible. Negligible oil production occurs when reservoir pressure is low enough such that an extra drop in pressure does not result in significant oil production or does not result in oil production that is economical.

As shown in the examples, the methods of the invention have been tested at reservoir conditions (e.g., reservoir pressures and temperatures), and by using reservoir rock samples and reservoir fluids. The examples demonstrate the efficiency of cyclic miscible hydrocarbon gas injection to recover oil from unconventional reservoirs and that hydrocarbon gas can penetrate into ultra-tight reservoir rock (i.e., unconventional reservoirs), develop miscibility with oil in the rock, and displace oil from pores in the rock. Without wishing to be bound by theory, it is thought that, if gas injection is continued during the soaking step to maintain pore pressure, oil recovery improves because gas penetrates deeper into the rock.

The invention can be further understood by the following non-limiting examples.

### EXAMPLES

Materials and Methods

Core-flooding tests were performed on three native-state reservoir core samples using different fluid systems comprising brine, crude oil, and gas and at different initial saturation conditions. Examples 1 and 2 used core samples with no fractures, and Example 3 used a composite core with a fracture that was filled with sand grains as proppant.

Core Samples

Three core samples were selected from two different wells. Table 1 provides the information and properties of the core samples used in this study.

TABLE 1
Petrophysical properties of the core samples used in this study.

| | | Length | Diameter | | Porosity^{a} |
|---|---|---|---|---|---|
| Example | Sample | (cm) | (cm) | Fractured | (%) |
| 1 | A | 8.407 | 3.78 | No | 3.5^{b} |
| | | | | | 3.3^{c} |
| 2 | B | 8.738 | 3.78 | No | 4.3 |
| 3 | B | 7.823 | 3.78 | Yes | 4.6 |
| 3 | C | 9.068 | 3.78 | Yes | 6.8 |

^{a}Measured at a net confining pressure of 2.07 MPa
^{b}Measured on a sister core
^{c}Obtained from X-ray images generated using a nano-CT scanner (no confining pressure was on the rock piece)

Prior to use, the core samples (hereafter referred to as Samples A, B, and C) were X-ray imaged with an X-ray macro-CT scanner with a resolution of 250 μm×250 μm×1000 μm to examine their integrity and homogeneity. No major heterogeneity was observed in the core samples.

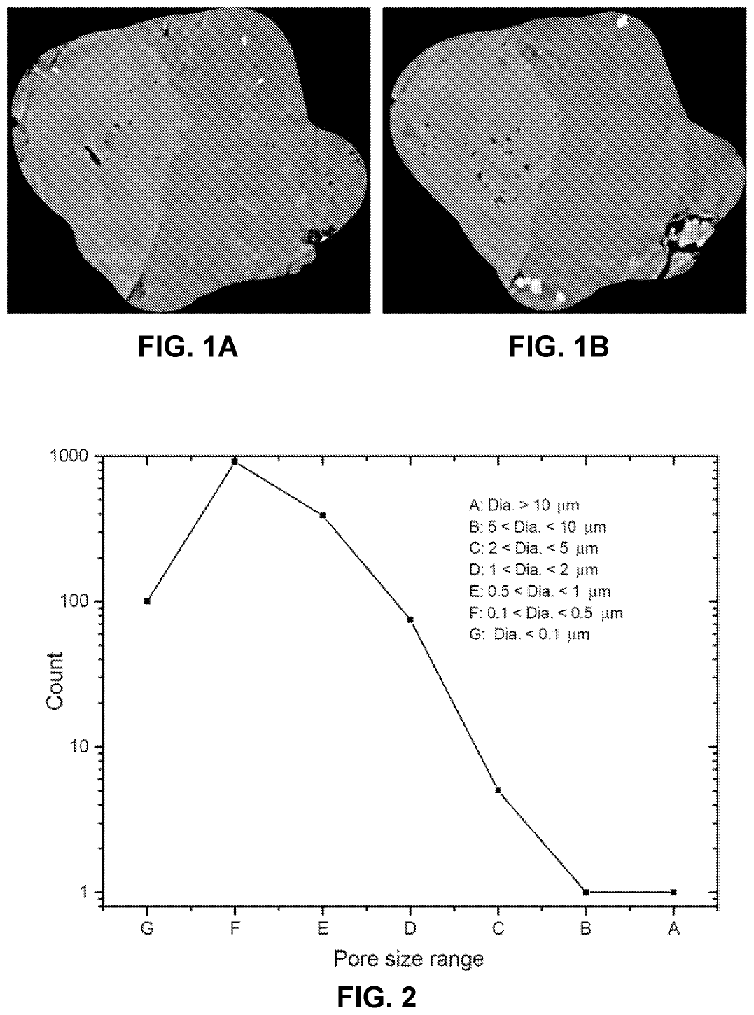

The porosity of Sample A was first estimated using a sister core drilled from the same depth in the same formation. The porosity of the sister core measured with Helium gas at a net c pressure of 2.07 MPa was 3.5%. The porosity of Sample A was also estimated by imaging a small piece of the core sample using a nano-CT scanner at a resolution of 64 nm. FIG. 1 shows two nano-CT images. Analysis of the images yielded a porosity of 3.3%.

The nano-CT images were also used to generate the pore-size distribution of the reservoir rock sample shown in FIG. 2. As shown in FIG. 2, a significant portion of the pore sizes range from 100 to 500 nm. These results are in agreement with a recent study characterizing a sister core sample. See M. Akbarabadi, S. Saraji, M. Piri, D. Georgi, M. Delshad, Nano-scale experimental investigation of in-situ wettability and spontaneous imbibition in ultra-tight reservoir rocks, Adv. Water Resour., 2017, 107, 160-179, which is incorporated by reference in its entirety.

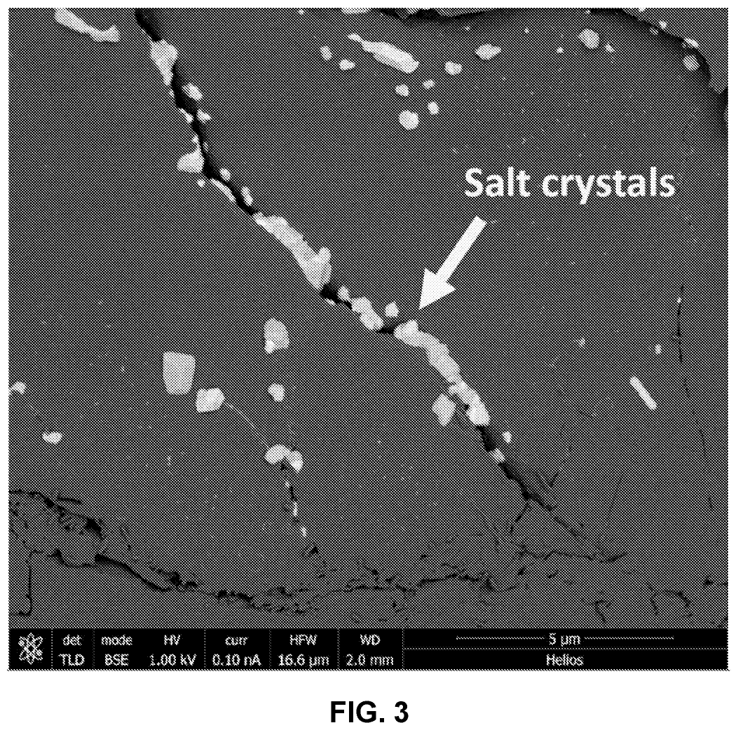

Rock pieces from different locations in the formation were selected to characterize pore elements and their conductivity using a scanning electron microscope (SEM). The SEM images revealed that all of the samples contained a significant amount of precipitated salt, which had covered the pore walls and either partially or fully plugged pore elements and had thus made parts of the pore space inaccessible to flow. Without wishing to be bound by theory, it is thought that salt may have precipitated throughout the coring process and core recovery as the pressure and temperature of the core samples decreased from reservoir conditions to ambient conditions resulting in water evaporation from hypersaline brine and leaving solid salt in the pore space. FIG. 3 exemplifies an SEM image taken from one of the samples. To remove the salt precipitates from Samples B and C, thereby mimicking the conditions of the reservoir, the samples were subjected to a cleaning process.

For example, Sample B was subjected to a cleaning process to extract salt precipitates. Sample B was placed in a core holder under 1.38 MPa net confining stress and saturated with methanol. Several pore volumes of methanol were then injected into the plug at a pore pressure of 5.17 MPa and a temperature of and 65° C. During the cleaning process, at a constant flow rate, pressure drops decreased across the core sample. In addition, the core sample's methanol permeability increased.

The salinity of the methanol effluent was monitored during the cleaning process. Methanol injection was continued until the salt concentration in the effluent reached a cutoff (approximately 5,000 ppm). Thereafter, the core sample was flushed with Helium gas and then vacuumed to remove bulk methanol and was then oven-dried at 65° C. until a stable weight was measured over two 48-hour readings. After drying, the porosity of Sample B was measured with helium under 2.07 MPa net confining pressure.

The pore volumes of Samples A and B in Examples 1 and 2 were approximately 4 cm³, and the pore volume of Sample C in Examples 1 and 2 was approximately 8.8 cm³.

To increase the pore volume, a composite core was used in Example 3. The composite core was created by using Sample B, which was previously utilized in Example 2, with Sample C. Before making the composite core, Samples B and C were first separately subjected to the cleaning process described above to remove salt precipitates. Samples B and C then had their porosities measured with Helium under 2.07 MPa net confining pressure (Table 1). The overall pore volume of the composite core sample was approximately 12.8 cm³.

Fluids

Examples 1-3 used different brine/crude oil/gas fluid systems. The brine phases of the Examples, summarized in Table 2, were prepared by dissolving NaCl, CaC12.6H2O, and MgC12 in distilled water.

TABLE 2
The composition of brines used in Examples 1, 2, and 3

| | NaCl | CaC1₂ 6H₂O | MgC1₂ | TDS^{a} |
|---|---|---|---|---|
| Example | (mg/L) | (mg/L) | (mg/L) | (mg/L) |
| 1 | 200,450.5 | 97,259.9 | 4,570.8 | 254,293.3 |
| 2 | 259,314.4 | 108,186.0 | 5,905.3 | 320,026.8 |
| 3 | 259,314.4 | 108,186.0 | 5,905.3 | 320,026.8 |

^{a}“TDS” stands for total dissolved solids based on anhydrous salts.

The crude oil phase of Examples 1 and 3 was stock tank oil. The stock tank oil was first passed through a 0.5-μm filter and then centrifuged at 5500 rpm for 2 hours to remove sand particles and brine. No water was detected in the stock tank oil. The asphaltene content of the stock tank oil after filtering and centrifuging, which was measured with n-heptane as a precipitant, was around 0.4 wt. %.

The crude oil phase of Example 2 was live oil. The live oil was reconstituted by (1) preparing a hydrocarbon gas mixture (60 mole % Cl, 22 mole % C2, 11.5 mole % C3, and 6.5 mole % C4 resembling the gas phase released from the crude oil) in a recombination cell; (2) adding stock tank oil in an amount determined from a gas oil ratio (GOR) of 600 SCF/STB to the recombination cell; (3) increasing the pressure of the recombination cell to a pressure above the bubble point pressure (Pb) of 13.79 MPa; and (4) shaking the recombination cell for two days. The Pb varies depending on oil composition and can be obtained through numerical simulation and compositional analysis of the in-situ crude oil. The Pb value also can be measured experimentally using PVT analysis using different methods. One method is a flash liberation test, wherein the pressure of the live oil suddenly drops to below the bubble point to ambient conditions. No gas is removed from the flash cell. Another method is a differential liberation test, wherein the pressure is dropped gradually (i.e., step by step) to ambient conditions. Liberated gas is removed from the cell at each step.

The gas phases of the Examples, summarized in Table 3, were prepared by adding the components gravimetrically to the recombination cell. To do so, the component with the lowest cylinder pressure was added first, and then the other components were added in order of increasing cylinder pressures. Using a gas chromatograph, the accuracy of the final gas mixtures was found to be within ±1% of their targeted compositions.

TABLE 3
The composition of the gas mixture used to inject
into the core sample in Examples 1, 2, and 3

| | Methane | Ethane | Propane | n-Butane |
|---|---|---|---|---|
| Example | (mole %) | (mole %) | (mole %) | (mole %) |
| 1 | 56.97 | 23.10 | 14.14 | 5.79 |
| 2 | — | — | 100.00 | — |
| 3 | 64.00 | 23.00 | 13.00 | — |

### Experimental Setup

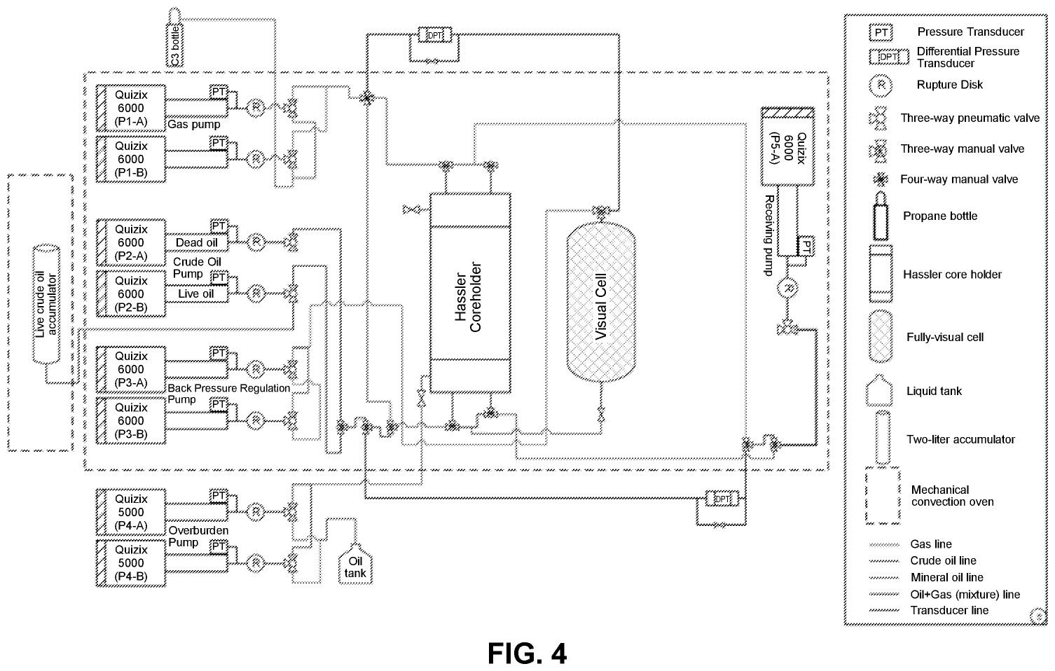

A core-flooding setup was designed and built to perform gas injection tests using live fluids at reservoir conditions. The setup comprised dual-cylinder pumps (Quizix), pressure transducers (Rosemount), a vertically-mounted Hassler-type core holder, a three-phase separator, live fluid recombination cells, and mechanical convection ovens. The lines used in the setup had a diameter of 0.3175 cm (⅛ inch) except the lines delivering produced fluids from the core sample to the separator. The lines delivering produced fluids had a diameter of 0.1587 cm ( 1/16 inch) to minimize the amount of liquid hold-up between the core holder and the separator. The pumps were assigned for injecting fluids (the stock tank oil, live oil, and gas) into the core samples, maintaining the pressure of the separator (the outlet pressure), and maintaining the overburden pressure. FIG. 4 shows a detailed schematic diagram of the core-flooding setup.

In Example 1, the separator did not have any windows to monitor possible oil production during gas injection and had to be opened and washed to collect produced oil. In Examples 2 and 3, the separator was replaced with a fully sapphire visual cell.

Example 1 used a Viton sleeve. However, this resulted in diffusion of the injected hydrocarbon gas phase into the confining fluid through the Viton sleeve. To avoid diffusion of the injected hydrocarbon gas phase into the confining fluid through the sleeve, the core samples in Examples 2 and 3 (after establishing initial water and oil saturations), were wrapped with Teflon tape, placed in heat shrink tube, and then wrapped with a layer of aluminum tape. Further, the Viton sleeve was replaced with a custom-built sleeve (manufactured through a customized method and using a specific type of AFLAS material (a copolymer of tetrafluoroethylene and propylene (TFE/P) material (7182TM)), which was wrapped with a layer of lead tape. These protective layers eliminated diffusion of hydrocarbon gas through the sleeve in the course of core-flooding.

Establishment of Initial Water and Oil Saturations

To establish an initial two-phase condition before gas injection, each core sample was first saturated with stock tank oil. To this end, each core sample was first weighed and was then mounted in the core holder under 1.38 MPa net confining pressure. This confining pressure was selected to enable access to as much pore space as possible when saturating the sample. After mounting in the core holder, the core sample was flushed with either CO₂ or methane for approximately one day to remove air and was then placed under strong vacuum for at least four days (vacuum was applied from both top and bottom for one day and from the top for three days).

The stock tank oil was then injected from both top and bottom of the core sample at constant pressure. The pressure of the injection pump was gradually increased to 3.45 MPa, and the pump continued injecting the oil under the 1.38 MPa net confining pressure. The sample was assumed to be fully saturated with crude oil when the flow rate of the pump decreased to almost zero. Subsequently, two pore volumes of the stock tank oil were injected into the core sample from the bottom at the 1.38 MPa net confining pressure (relative to the inlet pressure), and absolute permeability to the stock tank oil was measured. The permeability was not measured for Sample A, but the permeability of Sample B was measured as 3.02×10-18 m2 in Example 2 and 3.03×10-18 m2 in Example 3, and the permeability of Sample C was measured as 4.54×10-18 m2. After measuring the absolute permeability, the sample was depressurized slowly, taken out of the core holder, and weighed.

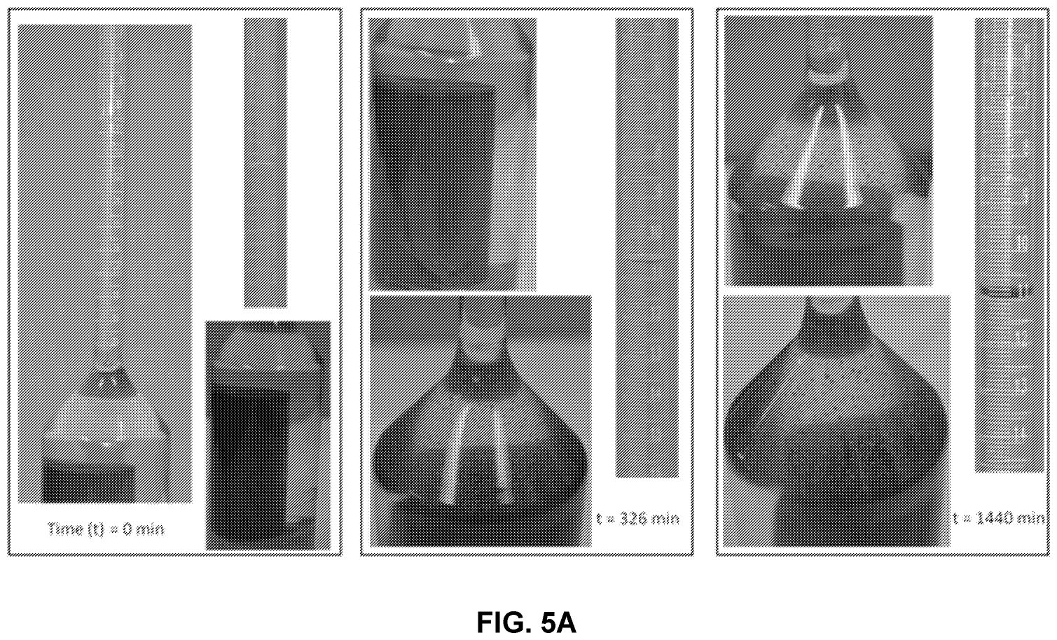

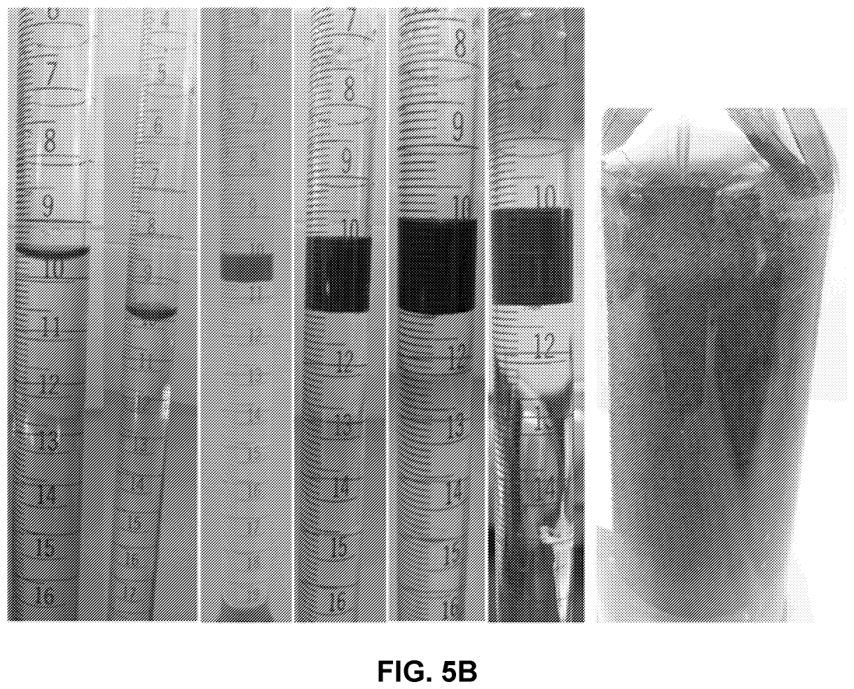

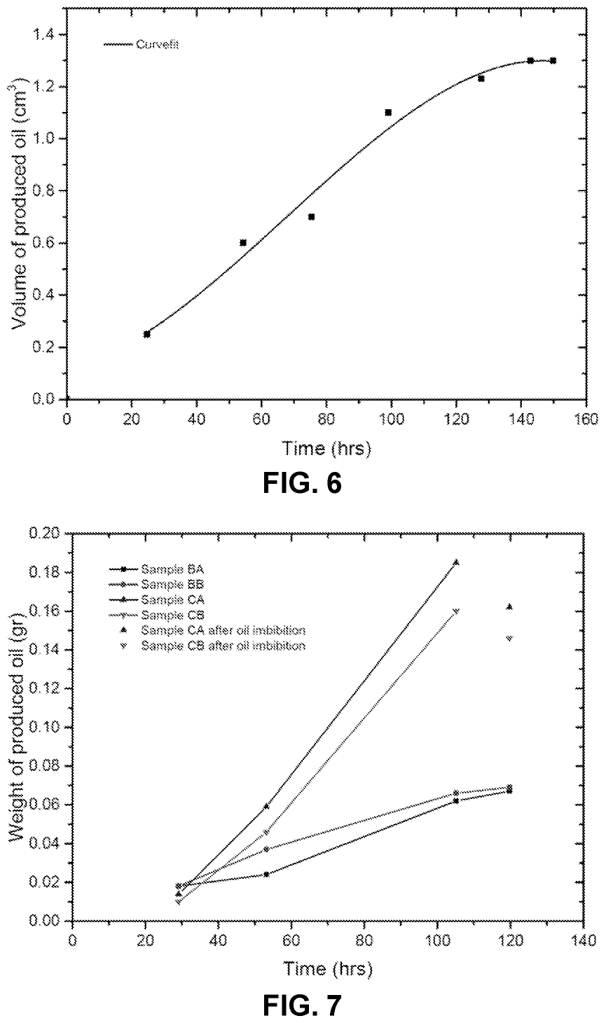

To establish initial water saturation in Examples 1 and 2, the core samples were separately placed in imbibition cells full of the synthetic formation brine. Because water saturation within a given formation may vary, two distinct ranges of initial water saturation were used in the Examples. Spontaneous imbibition of brine into Sample A started approximately 300 minutes after placing the core sample in the brine. The sample was left in the brine for approximately 1.5 days, during which oil production from the core sample was monitored volumetrically to estimate the water saturation in the core sample. After approximately 1.5 days, the core sample was taken out from the brine and weighed. The weight measurement indicated that an initial water saturation (Swi) of 11.50% had been established in Sample A. Production of oil from Sample B due to spontaneous imbibition of brine started similarly to that of Sample A. However, Sample B was left in the brine for approximately 6.5 days, which resulted in an initial water saturation (Swi) of 37.52% in Sample B. FIG. 5 shows Samples A and B during the spontaneous imbibition process. FIG. 6 shows the volume of oil produced from Sample B versus time during spontaneous imbibition.

After establishing initial water saturation, each core sample was placed back in the core holder and about two to three pore volumes of the stock tank oil were injected into the sample to ensure uniform distribution of the aqueous phase across the core sample. To avoid brine production, low flow rates (from 0.0005 cm³/min to 0.001 cm³/min) were used. When Sample A was weighed again, there was no change to the weight of the core sample after the second oil flood, indicating that no water had been produced. All the above-mentioned steps for the establishment of initial water and oil saturations were conducted at ambient temperature.

In Example 3, gas injection was performed in the presence of fractures. Thus, in Example 3, each core sample (i.e., each of Samples B and C) was first saturated with the stock tank oil and was then cut lengthwise into two pieces to create a fracture running from one end of the core sample to the other end. The cut was made such that the fracture in each core sample was approximately perpendicular to the bedding of the sample. The beddings were discerned from the X-ray images taken of the core samples as described above.

The pieces resulting from the cut are hereafter labeled as Samples BA, BB, CA, and CB. The desired initial water saturation of each piece was established by placing each individual piece in a separate imbibition cell filled with brine. To ensure that no one piece reached the desired initial water saturation in Example 3 (around 11%) earlier than the other pieces, each rock piece was taken out from the imbibition cell and stored in the stock tank oil until the other pieces reached similar initial water saturation. The weight of the piece(s) kept in the oil decreased during the time they were stored in the stock tank oil, indicating that the pieces had imbibed oil and expelled some brine. The density of oil was lower than the density of brine. FIG. 7 shows this change in weight: the last two points of Samples CA and CB show lower values. The variation in the established initial water saturations was considered in the final saturation for each piece. The final initial water saturation for Samples BA, BB, CA, and CB were 10.64, 9.51, 12.98, and 12.40%, respectively.

Creation of Composite Core with Propped Fractures

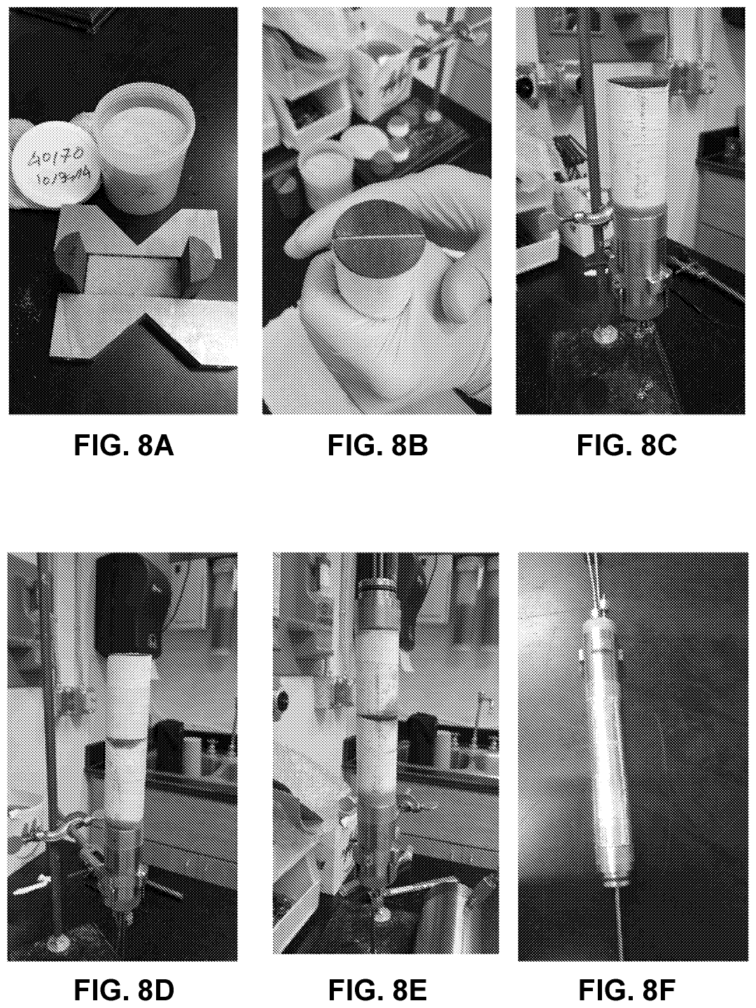

After establishing initial water saturation in Samples BA, BB, CA, and CB, a first piece of each main core sample (e.g., BB or CB) was covered with approximately three layers of proppant (40/70 sand). A second piece (e.g., BA or CA) was then placed on top of the first piece (e.g., BB or CB). The proppant was used to keep the fracture open in each core sample. Each core sample assembly (the assembled BA and BB and the assembled CA and CB) was subsequently wrapped with Teflon tape to avoid losing the proppants, but the inlet and outlet ends were not wrapped. Two paper filters wetted with crude oil were placed on both ends of each core sample to prevent proppant loss from the ends. Thereafter, Samples B and C were placed together such that the fracture plains of the plugs were perpendicular. The composite core, together with the end pieces of the core holder, was covered with shrink tube. A layer of aluminum tape was wrapped on the shrink tube. FIG. 8 exhibits the above-mentioned steps. After preparation, the composite core was scanned using the X-ray macro-CT scanner to image the fracture and proppant pack.

Experimental Procedure

After establishing initial water saturation, each core sample, together with the end pieces of the core holder, was wrapped with a protection layer (i.e., Teflon tape, heat shrink tube, aluminum tape) as described earlier and then placed in a core holder. After placing Sample A in the core holder in Example 1, the pore pressure was increased to 24.13 MPa by injecting the stock tank oil into both ends of the core sample while maintaining a net confining pressure of 6.89 MPa. Initial examination of the rock samples during helium porosity measurements indicated that the pore volumes of the plugs decreased by increasing the confining pressure.

During the rest of Example 1, the core outlet pressure was kept at 24.13 MPa and the confining pressure was set to 6.89 MPa above the outlet pressure (i.e., 31.02 MPa). When the initial pressure conditions were established in Example 1, the plug was flooded with the stock tank oil to measure effective oil permeability. The effective permeability to oil at Swi was 2.71×10-19 m2. Before gas injection, the temperature of the system was increased to I16° C. and then the lines upstream and downstream of the core sample were flushed with hydrocarbon gas mixture, bypassing the core sample, to remove excess oil left from the last oil flood and to exclude that amount from oil recovery calculations.

In Example 2, similar to Example 1, after placing Sample B in the core holder, the pore pressure and confining pressure were increased to 17.24 MPa and 62.05 MPa, respectively, by injecting the stock tank oil. These pressures were selected to be closer to reservoir conditions and to the conditions of the well from which Sample B had been obtained. Afterwards, the stock tank oil in the core sample was displaced with live oil at 17.24 MPa, and the temperature was increased to 80° C. The effective permeability to live oil at Swi was 6.91×10-19 m2. The live oil in the injection and production lines of the apparatus was removed with the hydrocarbon gas phase, bypassing the core sample, before the start of gas injection into the core.

In Example 3, the fractured composite core sample was put in the core holder under 6.89 MPa confining pressure, and then the proppant pack was lightly vacuumed and saturated with the stock tank oil. During the stock tank oil injection, the pore pressure, confining pressure, and temperature were slowly increased to 17.24 MPa, 62.05 MPa, and 96° C., respectively. Before gas injection, the stock tank oil in the injection and production lines was flushed out with the gas phase at 17.24 MPa by passing the core sample. Approximately 4 fracture pore volumes of gas were also injected into the fracture to displace oil present in the proppant pack. The total fracture volume and the average fracture aperture were determined by visualizing the CT images using Avizo software. The average fracture apertures and fracture pore volumes of sample B and C were 1.46 and 1.22 mm and 1.94 and 1.96 cm³, respectively. Therefore, the total fracture pore volume of the composite core was 3.9 cm³. Porosity of the proppant pack was assumed to be 50%. It was impractical to measure the porosity of the proppant pack with the CT scanner because the core holder selected for this Example was made of Hastelloy (i.e., non-scannable) to tolerate 62.05 MPa confining pressure. Thus, the fractured composite core was scanned without the core holder under no confining pressure.

The gas injection steps of the three core-flooding Examples were carried out using a methodology known as huff-n-puff (also referred to herein as an inject-soak-produce procedure). A huff-n-puff cycle included injecting the gas phase into the core sample and building up the pore pressure to a pre-specified pressure, soaking the gas phase with the resident fluids in the pore space for a given period, and then producing oil from the core by reducing the pore pressure to a specific pressure.

In each Example, the injection-soak-production cycle was repeated three to four times, and then the last cycle was followed by a blow-down process to ambient conditions. Although, a separator was located at the outlet of the core holder to collect all the effluent throughout the gas injection process, the final oil recovery in each Example was calculated based on the weight of the core before and after gas injection (after establishing the initial two-phase condition in the core and after the blow-down, respectively). This was done because the amount of oil produced and collected in the separator increased due to expansion as well as the dissolution of the gas phase. Therefore, it was impractical to calculate the oil recovery volumetrically.

### Example 1

To start the gas injection process in Example 1, the outlet of the core (i.e., the bottom of the core holder) was isolated, and the gas phase was injected from the top of the core at an average flow rate of 0.002 cm³/min for approximately 43 hours, which increased the inlet pressure from its initial value of 24.13 MPa to 26.20 MPa. The gas was initially injected at a flow rate of 0.005 cm³/min, but this flow rate resulted in a rapid increase of inlet pressure because of the ultra-low permeability of the core sample. Thus, the gas flow rate was adjusted to have a gradual build-up in the inlet pressure. Thereafter, the gas pump was set at constant pressure to maintain the inlet pressure at 26.20 MPa, and the core sample was soaked for approximately 72 hours. During the injection and soak steps, a total of 1.82 pore volumes of gas were injected into the core holder.

Although the gas phase could have been compressed during the pressure buildup, the amount of injected gas was high. Without wishing to be bound by theory, it is thought that the high amount of gas injected was partly caused by diffusion of the gas phase to the overburden fluid through the Viton sleeve.

After soaking, the core holder was isolated from the gas injection pump, the separator was pressurized with nitrogen gas to 26.20 MPa, and then the outlet valve between the core holder and the separator was opened. The change in the pressure of the separator was minimal, which indicated that the outlet pressure of the core had also increased from its initial value of 24.13 MPa to 26.20 MPa during gas injection.

Without wishing to be bound by theory, it is thought that increase in the pore pressure and the outlet of the core indicated that the hydrocarbon gas phase could penetrate the ultra-low-permeability core sample. During the production step, the outlet pressure of the core was gradually decreased to 24.13 MPa within 7 hours by retracting fluids from the bottom of the core holder using the back pressure pump.

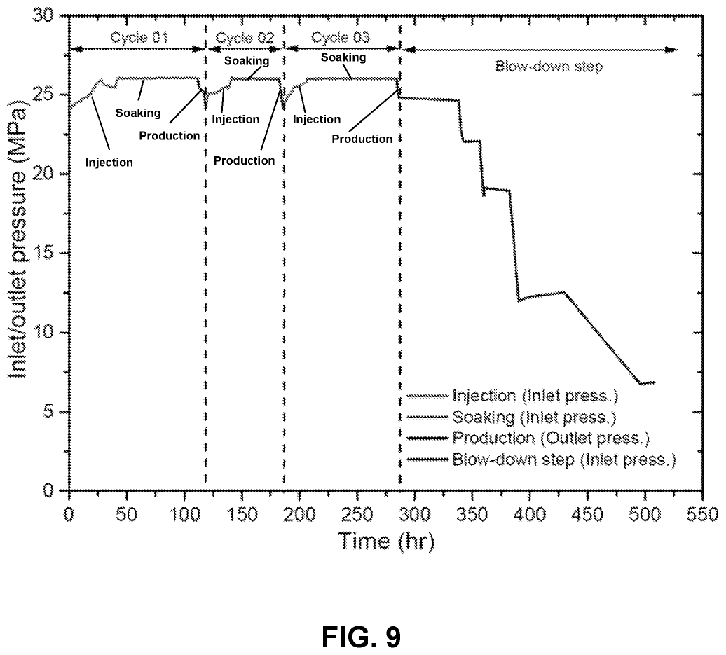

After the production step of the cycle, the outlet value between the core holder and separator was closed and the inlet valve was opened. The cycle of injection-soak-production was repeated twice using a similar procedure. The variations of the pressure throughout the three cycles are shown in FIG. 9.

After completion of the third cycle, the blow-down process was started by reducing the pressure of the separator (i.e., the outlet of the core plug) in steps from 24.13 MPa to 6.89 MPa. When the pore pressure reached 6.89 MPa, the gas injection was stopped and the pressure and temperature of the system were reduced to ambient conditions to weigh the core sample and calculate oil recovery.

Oil recovery was calculated based on the weight of the core sample before gas injection and after gas injection. The difference in weight indicated that 84% of the original oil in place was recovered due to gas injection. Since the separator used in Example 1 was not visual, the lines and separator were washed with solvents after disassembling the setup. The effluent was brown implying that oil had been produced during Example 1. X-ray images of the core sample after Example 1 did not show any visible changes in the structure of the rock.

In Example 1, it was assumed in the calculations that the pore space originally had almost no liquids (i.e., brine and oil), and therefore no liquid was lost during vacuuming the core plug and the weight of the core remained unchanged. This assumption was partly supported by the nano-CT images of a small piece of the rock that showed a small amount of oil and no brine in the pore space. However, it is possible that this observation does not apply to all parts of the core sample.

In Example 1, it was assumed that stressing the core plug has the same impact on initial water and oil saturations. However, because water saturation in Example 1 was low, it is possible that squeezing the core sample may have resulted in expelling oil more than brine.

Example 1 demonstrates that hydrocarbon gas can penetrate ultra-low-permeability rock and produce oil from the rock.

### Example 2

The procedure used in Example 2 was similar to that of Example 1, with some differences. First, the oil and gas phases in Example 2 were different from those used in Example 1. For instance, Example 2 used pure propane, which is liquid at the prevailing experimental conditions (e.g., reservoir conditions). Second, the closed separator of Example 1 was replaced with a fully visual cell in Example 2. Third, the gas injection process in Example 2 was started at 17.24 MPa, which is closer to the pressure of formation after primary production and before gas injection.

After closing the outlet of the core holder, propane was injected from the top to increase the inlet pressure from 17.24 MPa to 26.20 MPa. When the inlet pressure reached 26.20 MPa, the inlet pressure was maintained using a pump that supplied more propane into the core sample. Maintaining inlet pressure during soaking was important because, without wishing to be bound by theory, it is thought that small amounts of propane invaded the core sample during the injection step given propane's liquid state at the prevailing pressure and temperature conditions. The soak step was continued until the flow rate of the pump reached almost zero, which indicated that the outlet pressure of the core sample had increased to 26.20 MPa and that no more propane dissolution in the crude oil occurred.

After soaking, the oil production step was commenced. To this end, the separator was pressurized with nitrogen gas to 26.20 MPa, the inlet valve was closed, and the outlet valve between the core holder and the separator was opened. The change in the pressure of the separator was minimal, indicating that the outlet pressure had increased to 26.20 MPa. The pressure of the separator connected to the outlet of the core was gradually decreased in 3 hours to 24.13 MPa.

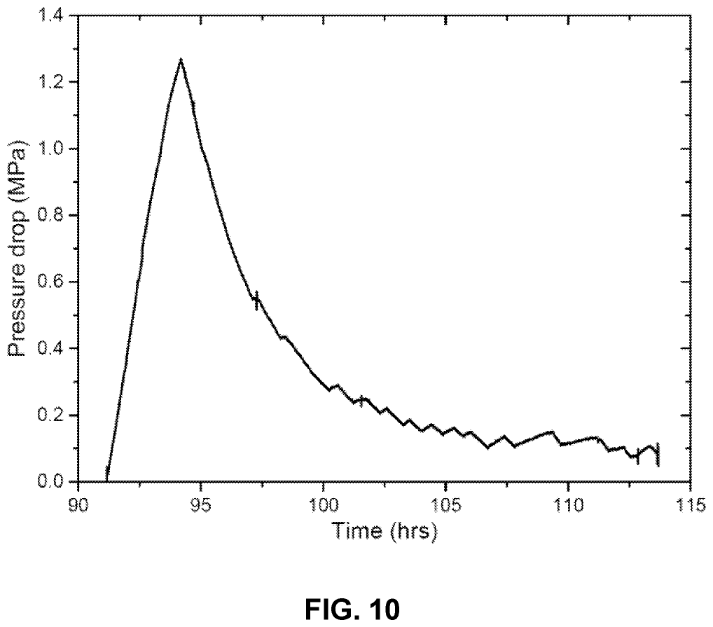

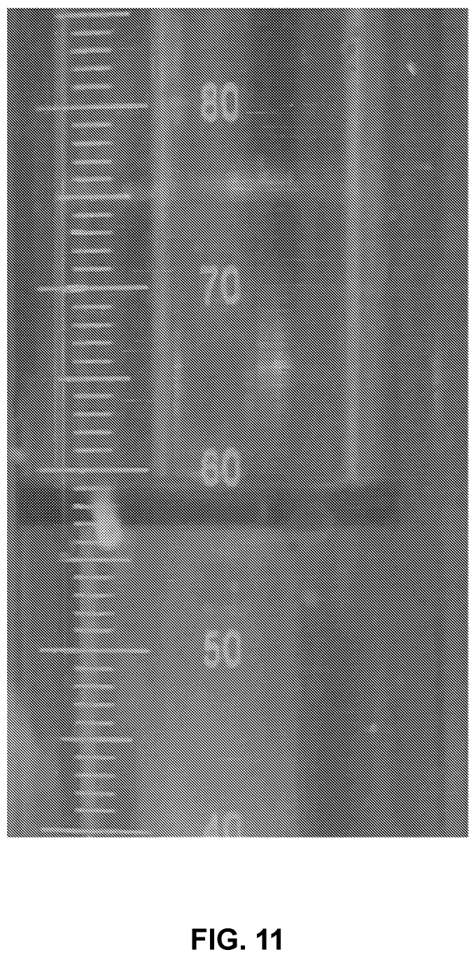

The production step was continued until both the retraction flow rate of the pump (maintaining the separator pressure at 24.13 MPa) and the pressure drop across the core sample reached almost zero. Variations of the pressure drop across the core sample during the production step of the first cycle versus time are plotted in FIG. 10. The graph starts at a pressure drop of zero because the pressure of both the inlet and outlet was 26.20 MPa before the production step. No oil was observed in the separator at the end of the first cycle. Without wishing to be bound by theory, it is thought that oil may have entered the production lines but had insufficient volume to reach the separator. The second and third injection-soak-production cycles were carried out using the same procedure. No oil was seen in the separator until the production step of the third cycle, when oil entered the visual cell, as shown in FIG. 11. This oil was the first direct evidence of production before blow-down. Thus, Example 2 demonstrated that oil was produced as a result of the injection-soak-production scheme.

After all the three cycles were completed, the blow-down process was started by reducing the pressure of the separator in steps from 24.13 MPa to 6.89 MPa. When the pore pressure reached 6.89 MPa, the pressure and temperature of the system were reduced to ambient conditions to take out Sample B and measure its weight.

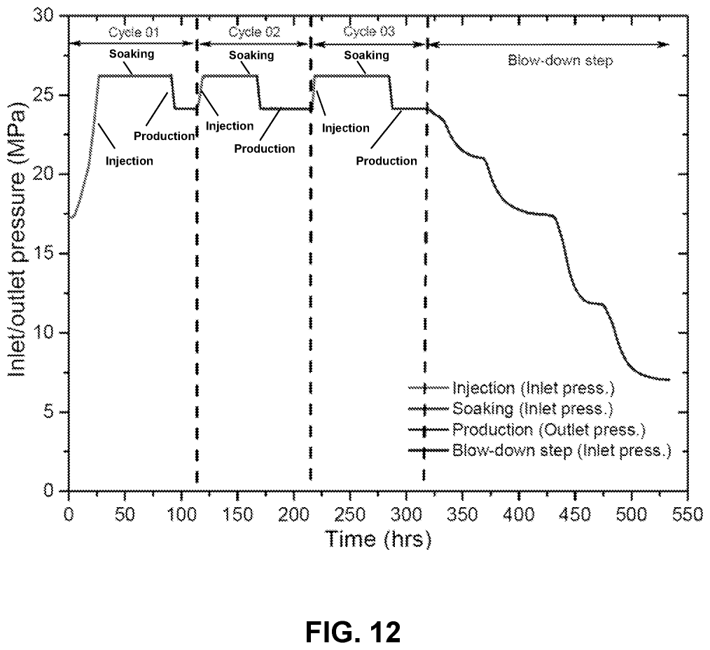

FIG. 12 shows variations of the inlet/outlet pressures over time throughout Example 2 before the temperature and pressure were decreased to atmospheric conditions.

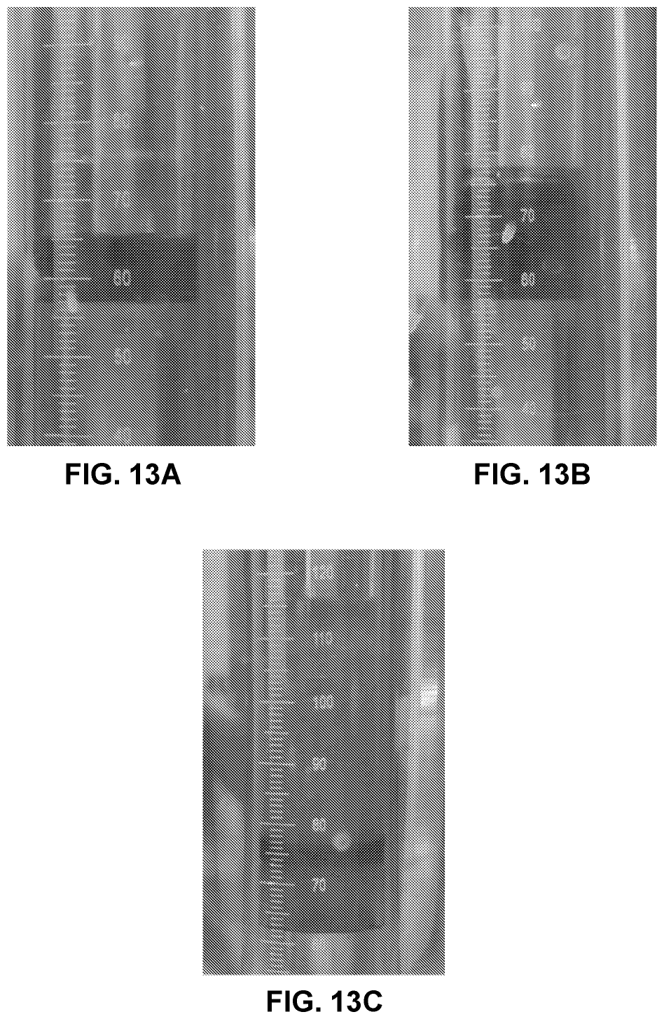

FIGS. 13A-C illustrate the separator at various stages of the blow-down process. The volume of oil in the separator did not represent the oil volume left the core. Instead, the volume of oil in the separator combined oil production, oil and propane expansion, and propane dissolved in the oil. The latter caused the color of the oil to gradually become lighter.

After taking the core sample out of the core holder, the core sample was imaged using an X-ray macro-CT scanner, which revealed that no damage induced during the core-flooding test.

The core sample was also weighed. Comparison of the core sample's weight before and after Example 2 revealed that the ultimate oil recovery factor was between 48.51%-75.56%. The higher value, 75.56%, assumes that the core sample produced no brine (the final water saturation remained 37.52%) and that weight loss of the core sample resulted from oil production.

However, it was noticed that, at the end of the test, the initial volume of brine in the separator had increased a little. While this excess brine might have been in the production lines at the start of the gas injection process (the separator filled with brine and nitrogen was connected to the outlet (bottom) of the core holder), it could also have been produced from the core sample due to the high water saturation in Example 2.

The lower value, 48.51%, assumes that the core sample produced all of the brine found in the separator after Example 2. However, it is possible that this brine was in the production lines before the gas injection process.

Example 2 proves that hydrocarbon gas can penetrate ultra-low-permeability rock and produce oil from the rock.

### Example 3

The procedure used in Example 3 was similar to that of Examples 1 and 2, with some differences. First, in Example 3, a composite core sample with a fracture was used, as described above. After the initial conditions in the composite core were established and before the gas injection process was started, the oil in the proppant pack in the fracture was displaced with the hydrocarbon gas at 17.24 MPa. Thereafter, the outlet of the core holder was closed and the hydrocarbon gas was injected from the top to increase the pressure of the core sample from 17.24 MPa to 27.58 MPa.

The range of pressurization and depressurization in Example 3 was between 20.68 MPa and 27.58 MPa to mimic operational conditions in the field. Due to the high conductivity of the fracture (and the proppant pack) running from one end of the composite core to the other end, gas initially invaded the fracture and therefore pressure of the fracture increased sooner than the pressure of the matrix. Thus, the injection step of all the cycles was conducted in approximately one hour.

Unlike Examples 1 and 2, the gas injection pump in Example 3 was disconnected from the core sample and no more gas was provided during the soak step. In Examples 1 and 2, injecting gas during soaking could be justified because there was no fracture filled with the gas across the core. Thus, in Examples 1 and 2, it was thought that the gas injected during the injection step was sufficient to increase only the inlet pressure due to ultra-low permeability of the matrix.

During soaking, while gas dissolved in the crude oil present in the adjacent matrix (i.e., close to the fracture) and moved further into the core sample (initially at 17.24 MPa), the initial 17.24 MPa pressure of the fracture dropped. In the first cycle, pressure drop occurred rapidly due to a small leak from a fitting. In subsequent cycles, the fitting was fixed, and pressure drops occurred more slowly. The soak step in all cycles was conducted for approximately 48 hours.

To start the production step, the pressure of the separator was increased to the pressure of the fracture and then the core holder was connected to the separator. During the production step, the pressure of the separator was gradually reduced to 20.68 MPa in 3 hours and then was maintained for a period of time sufficient (approximately 14-16 hours) to further matrix-fracture interactions until the flow rate of the separator pump was almost zero. No gas was injected into the fracture while the pressure was reduced. When the pressure reached 20.68 MPa, the fracture was flushed with four fracture pore volumes of the hydrocarbon gas to collect oil produced from the core sample in the separator.

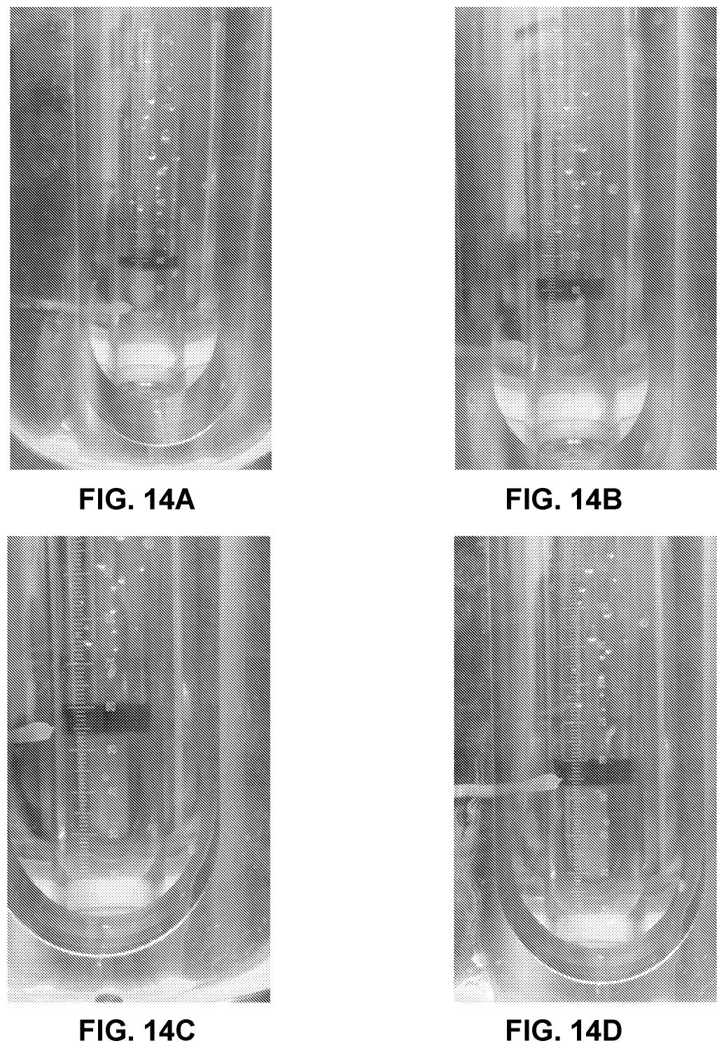

FIG. 14A shows the separator at the end of the first cycle. FIG. 14A demonstrates that miscible hydrocarbon gas injection into a fractured core sample from the formation resulted in oil production.

Example 3 was continued by conducting three more cycles. As shown in FIGS. 14B-14D, the rate of production decreased in the subsequent cycles due to the decrease in the oil present in the matrix.

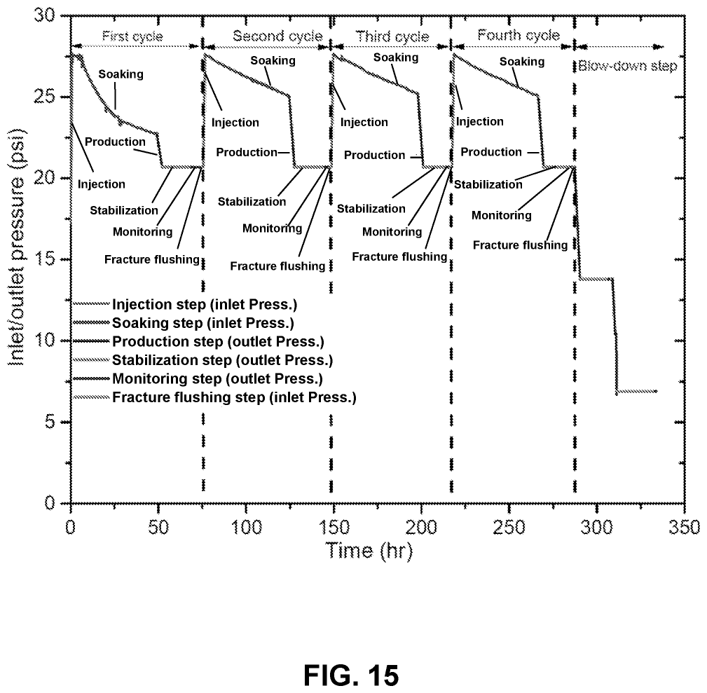

After completion of the fourth cycle, the blow-down process was started by reducing the pressure of the separator from 20.68 MPa to 13.79 MPa, then to 6.89 MPa, and finally to atmospheric pressure. In each step, the separator pressure was decreased gradually and maintained for a period sufficient to further matrix-fracture interactions. At the end of each pressure reduction step, the fracture was flushed with four fracture pore volumes of gas to collect any produced oil in the separator. The variations of the fracture pressure with time throughout Example 3 is shown in FIG. 15.

At the end of Example 3, the composite core sample was removed from the core holder and imaged using an X-ray macro-CT scanner, which revealed new macro-fractures perpendicular to the main fracture. Without wishing to be bound by theory, it is thought that the new fractures were caused by the non-uniformity of the proppant pack across the core and the high confining pressure (62.05 MPa) applied in Example 3.

The composite core sample was taken apart to separate the proppant pack and weigh each piece separately. Using the weight of each piece before and after Example 3, and assuming no brine was produced during Example 3, the oil recovery factors of Sample BA, BB, CA, and CB were 36.85, 32.82, 6.89, and 10.15%, respectively.

The results and observations made in Example 3 were important because the formation is heavily fractured, including natural and hydraulic fractures, and most oil production should be obtained from the interaction of the gas phase in the propped or unpropped fractures with the crude oil in the matrix. Example 3 proved the feasibility of miscible hydrocarbon gas injection into the formation.

The foregoing description and examples have been set forth merely to illustrate the invention and are not meant to be limiting. Since modifications of the described embodiments incorporating the spirit and substance of the invention may occur to persons skilled in the art, the invention should be construed broadly to include all variations within the scope of the claims and equivalents thereof.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains.

References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A method for evaluating the efficiency of oil recovery from an unconventional reservoir rock sample using a cyclic inject-soak-produce scheme, comprising the steps of:
(a) scanning the rock sample;
(b) characterizing the rock sample by imaging the rock sample using a nano-CT scanner and/or scanning electron microscopy (SEM);
(c) cleaning the rock sample using a flow-through technique with solvent selected from the group consisting of methanol, isopropanol, toluene, and mixtures thereof;
(d) saturating the rock sample with crude oil; and
(e) establishing initial water saturation in the rock sample.

2. The method of claim 1, wherein the rock sample is saturated with crude oil by injecting oil into the rock sample at a flow rate from 0.0001 cm³/min to 0.1 cm³/min.

3. The method of any of claims 1-2, wherein the rock sample is saturated with crude oil at a temperature of 18° C. to 60° C. and a pressure of 1 MPa to 10 MPa.

4. The method of any of claims 1-3, wherein the initial water saturation of the rock sample is established by immersing rock samples in brine.

5. The method of any of claims 1-4, further comprising the step of (f) performing a huff-n-puff gas injection on the rock sample, wherein the huff-n-puff gas injection on the rock sample is performed by (i) injecting gas into the rock sample; (ii) providing a time period to soak the rock sample with its fluid contents; (iii) producing production fluids, such as oil, water, and gas; (iv) optionally repeating steps (i) through (iii); and (v) decreasing pore pressure until oil production is negligible.

6. The method of claim 5, wherein the gas that is injected into the rock sample is selected from the group consisting of methane, ethane, propane, n-butane, isobutane, and mixtures thereof;
wherein the gas that is injected into the rock sample further optionally comprises CO₂; and
wherein the gas that is injected into the rock sample may comprise trace amounts of other components selected from the group consisting of nitrogen, pentane, hexane, heptane, octane, nonane, hydrocarbons with ten or more carbon atoms, and mixtures thereof.

7. The method of any of claims 1-6, further comprising the steps of:
(i) fracturing the unconventional reservoir rock sample,
(ii) creating a composite core from the rock sample; and
(iii) performing a huff-n-puff gas injection on the composite core.

8. The method of claim 7, wherein the rock sample is fractured by cutting the rock sample or by using a Brazilian test; and
wherein the composite core is created by placing two or more rock samples together.

9. The method of claim 7, wherein the huff-n-puff gas injection on the composite core is performed by (i) injecting gas into the composite core; (ii) providing a time period to soak the composite core with its fluid contents; (iii) producing production fluids, such as oil, water, and gas; and (iv) optionally repeating steps (i) through (iii); and (v) decreasing pore pressure until oil production is negligible.

10. The method of claim 9, wherein the gas that is injected into the composite core is selected from the group comprising methane, ethane, propane, n-butane, isobutane, CO₂, and mixtures thereof;
wherein the gas that is injected into the composite core may comprise trace amounts of other components selected from the group consisting of nitrogen, pentane, hexane, heptane, octane, nonane, hydrocarbons with ten or more carbon atoms, and mixtures thereof.

11. The method of any of claims 1-10, wherein the rock sample is scanned using an X-ray macro-CT scanner.

12. A method for improving the recovery of oil from an unconventional reservoir rock sample, comprising:
(a) establishing initial oil-brine saturation conditions in the rock sample;
(b) injecting a gas phase into the rock sample, thereby increasing a pore pressure;
(c) soaking the rock sample with its fluid contents from 1 h to 300 h;
(d) recovering oil using a huff-n-puff gas injection;
(e) monitoring the pore pressure until the pore pressure is stabilized;
(f) optionally repeating steps (b) through (e); and
(g) decreasing pore pressure until oil production is negligible.

13. The method of claim 12, wherein the initial oil-brine saturation conditions in the rock sample are established by:
(i) first saturating the rock sample with brine and then displacing brine with oil to establish initial water saturation at a temperature of 18° C. to 60° C. and a pressure of 0.1 MPa to 10 MPa; or
(ii) first saturating the rock sample with dead oil and then immersing the rock sample into brine in an imbibition cell to establish initial water saturation at a temperature of 18° C. to 60° C. and a pressure of 0.1 MPa to 10 MPa.

14. The method of any of claims 12-13, wherein the gas phase injected into the rock sample is selected from the group comprising methane, ethane, propane, n-butane, isobutane, CO₂, and mixtures thereof; and
wherein the gas injected into the rock sample may further comprise trace amounts of other components selected from the group consisting of nitrogen, pentane, hexane, heptane, octane, nonane, hydrocarbons with ten or more carbon atoms, and mixtures thereof.

15. The method of any of claims 12-14, wherein the huff-and-puff gas injection comprises decreasing the pore pressure to a pressure above a live oil bubble point pressure.

16. The method of any of claims 12-15, wherein the pore pressure is stabilized by:
(i) monitoring the pressure when the gas flow is stopped; or
(ii) monitoring the flow rate when pore pressure is kept constant throughout retracting the fluid from the production line by using a back pressure regulation pump under a constant pressure mode.

17. A method for improving the recovery of oil from an unconventional reservoir, comprising:
(a) injecting a gas phase into the unconventional reservoir, thereby increasing a reservoir pressure;
(b) soaking the unconventional reservoir with its fluid contents for 5 days to 35 days;
(c) recovering oil using a huff-n-puff gas injection;
(d) optionally repeating steps (a) through (c); and
(e) decreasing reservoir pressure until oil production is negligible.

18. The method of claim 17, wherein the gas phase injected into the unconventional reservoir is selected from the group comprising methane, ethane, propane, n-butane, isobutane, CO₂, and mixtures thereof; and
wherein the gas injected into the unconventional reservoir may further comprise trace amounts of other components selected from the group consisting of nitrogen, pentane, hexane, heptane, octane, nonane, hydrocarbons with ten or more carbon atoms, and mixtures thereof.

19. The method of any of claims 17-18, wherein the huff-and-puff gas injection comprises decreasing a pore pressure to a pressure above a live oil bubble point pressure.

20. The method of any of claims 17-19, further comprising establishing initial oil-brine saturation conditions in the rock sample, wherein establishing initial oil-brine saturation conditions in the rock sample comprises:
(i) first saturating the rock sample with brine and then displacing brine with oil to establish initial water saturation at a temperature of 18° C. to 60° C. and a pressure of 0.1 MPa to 10 MPa; or
(ii) first saturating the rock sample with dead oil and then immersing the rock sample into brine in an imbibition cell to establish initial water saturation at a temperature of 18° C. to 60° C. and a pressure of 0.1 MPa to 10 MPa.

21. The method of any of claims 17-20, further comprising monitoring the pore pressure until the pore pressure is stabilized; wherein monitoring the pore pressure comprises:
(i) monitoring the pressure when the gas flow is stopped; or
(ii) monitoring the flow rate when pore pressure is kept constant throughout retracting the fluid from the production line by using a back pressure regulation pump under a constant pressure mode.
